(19) United States
(12) Patent Application Publication
(10) Pub. No.: US 2024/0008768 A1
(43) Pub. Date: Jan. 11, 2024

(54) IMPLANTABLE CATHETER FOR MEDICATION DELIVERY AND ANALYTE SENSING

(71) Applicant: PhysioLogic Devices, Inc., Alpine, CA (US)

(72) Inventors: Peter C. Lord, Alpine, CA (US); Donald M. Cohen, Irvine, CA (US); Pedrum Minaie, Burbank, CA (US)

(21) Appl. No.: 18/035,778

(22) PCT Filed: Nov. 8, 2021
(86) PCT No.: PCT/US2021/058438

§ 371 Date: May 8, 2023

Related U.S. Application Data
(60) Provisional application No. 63/204,997, filed on Nov. 6, 2020.

(51) Int. Cl. A61B 5/145; A61B 5/00; A61M 25/00

(52) U.S. Cl. CPC A61B 5/14503; A61B 5/14532; A61B 5/4839; A61M 25/007; A61M 25/0045; A61M 2025/0073

(57) ABSTRACT

An implantable catheter for medication delivery and analyte sensing is provided. In embodiments, a permanent catheter portion for use in an implanted delivery device, includes a permanent tube with an inner lumen, a sleeve attached to the permanent tube, the sleeve having outer coating that is at least one of: non-inflammatory or preventative of foreign body response. The permanent tube further includes an opening to receive a distal catheter portion that extends into the sleeve, such that the inner lumen of the permanent tube is fluidly connected to a distal inner lumen of the distal catheter portion.

### CROSS-REFERENCE TO RELATED APPLICATIONS

This application claims the benefit of U.S. Provisional Patent Application No. 63/204,997, filed on Nov. 6, 2020, the entire disclosure of which is hereby incorporated herein by reference.

### TECHNICAL FIELD

The present invention relates generally to catheters implanted in the body for the delivery of medication, or for measuring analytes, or for both purposes in combination. In particular, systems and methods are disclosed for long term catheter patency, efficient and rapid distribution of medication, as well as transmittal of analytes to sensors mounted on a catheter.

### BACKGROUND OF THE INVENTION

Implantable drug delivery systems include an implantable pump, one or more medication catheters, and optionally one or more sensors. One such example is an implantable automatic insulin delivery system, such as the ThinPump™, developed by PhysioLogic Devices, Inc. (“PLD”), which transforms the treatment of insulin requiring diabetes. PLD's technology automatically controls glucose through a state-of-the-art implantable insulin pump paired with a glucose sensor. Using the PLD device, normal insulin and glucose physiology is restored because the insulin is delivered deep in the abdomen for uptake by the liver. https://physiologicdevices.com/.

However, conventional implanted medication catheters that deliver insulin, and implantable sensors that measure glucose are not able to reliably survive for the full battery life of the implantable pumps in their respective systems. Thus, a patient with such a medication delivery system must undergo surgery for a replacement catheter prior to the end of the battery life of the implantable pump. A long-life catheter would prevent this extra surgery. Likewise, implanted sensors encapsulate in less than a year, making it necessary to replace them frequently, often before the sensing chemistry has degraded.

The reason implanted medication delivery catheters, such as intraperitoneal and subcutaneous insulin delivery catheters, for example, have a limited longevity is due to the problems of encapsulation and lumen blockage. Similarly, implanted glucose sensors, such as, for example, intraperitoneal and subcutaneous glucose sensors, have a limited longevity due to the buildup of a tissue capsule around the sensor that limits the diffusion of analyte and reactants (including oxygen) to the sensor.

What is needed in the art are solutions to these problems.

### SUMMARY OF THE INVENTION

Methods for increasing the operational life of an implanted catheter and improving the kinetics of medication delivery and analyte diffusion to catheter mounted analyte sensors such as glucose sensors are presented. In embodiments, the dispensing area of the catheter may be increased and the locations of the dispensing holes or porosity are widely distributed to achieve three goals. First, to spread the distribution of medication over a large area, so that instead of a spherical depot there is an increased area for distribution of medication. Second, to provide for the openings to be sufficiently remote from each other to prevent distribution into a common depot. Keeping the distance between widely distributed assures a reduced likelihood of a tissue build up that would affect adjacent openings. Third, to introduce medication into the largest possible area of tissue to speed, for example, insulin dilution and rapid absorption.

It is desirable to mount sensors, such as, for example, glucose sensors, on the sidewall of catheters. The design of an extended life catheter with distributed openings will extend the life of analyte sensors on the sidewall by the same mechanism. By distributing the sensors widely over the surface of the catheter, the effect of a possible encapsulation of one sensor will not affect the operation of any other sensor. In embodiments, this improves the reliability and operational life of the sensor system and enhances system reliability, because the system depends upon reliable information from the sensors.

In embodiments, each of the openings of the catheter are designed to provide equal medication flow by the design of the catheter fluid path. For example, equal flow may be achieved by one of the following two methods. First, in the case of a simple catheter with a single and relatively small diameter lumen and a series of side holes, the holes may have different sizes to match different pressures along the catheter. For example, holes may be smaller at the proximal end of the catheter and larger at the distal end to be in proportion to the higher pressure at the proximal end and lower pressure at the distal end of the catheter (as there is always a pressure drop during fluid flow over the length of the catheter, as given by the Poiseuille-Hagen equation). This is reflected in the size of holes shown in FIGS. 1, 2, 6 and 7, for example.

Second, in the case of a catheter with a relatively large lumen, equal flow may be achieved by using smaller holes or openings with restrictive elements, such as a porous plug, for example, that are significantly more restrictive than the large lumen. In an extreme example case, a large lumen could reside inside a full-length porous component that is very restrictive. Because the restrictiveness of the full-length porous component is significantly greater than the lumen, medication infuses evenly (flow/unit of area) over the full length of the porous component.

In embodiments, such a component (or sleeve) may ideally be shaped to prevent medication accumulation in response to pressure as well as for maximum surface area for wide distribution of medication. In embodiments it may be, for example, soft and flexible to avoid irritation of tissue, and, for example, constructed of materials that do not provoke a foreign body response. Alternatively, or even additionally, it may be coated with materials that prevent tissue build up or foreign body response, such as, for example, dexamethasone.

It is noted that the delivered bolus volumes of medication for implantable catheters are small, generally between 0.05 and 2.0 microliters (μL). The catheter must dispense this amount of liquid in a consistent manner and not expand or otherwise accumulate the medication due to catheter compliance in reaction to blockage or pressure at the catheter openings. (“Compliance” is the capacity of the catheter to accommodate the sudden change in contents without delivery to the outlet. There may always be some compliance, but the compliance should not interfere with the ultimate timely, consistent, even and desirable delivery flow volume.) In embodiments, the catheter is constructed of an hydraulically rigid material, such that there is no dimensional change in the catheter with pump stroke pressure. For example, the catheter may be constructed as a cylinder, or as a flat paddle shape, using flexible materials that do not expand or otherwise change their volume with the expected medication delivery pressures, thus providing consistent delivery of these small volumes over a large area.

The pressure at outlet of a pump mechanism into a high flow resistance could be of psi, but typically at the outlet, the pressure is very low; less than 1 psi. The objective is consistent, near uniform delivery over a wide field so that absorption is rapid. Large catheter compliance would protract the delivery time (much as a capacitor in a resistive electronic circuit can store energy and create a time element in what otherwise would be an instantaneous process) and so is not desirable. Some compliance is acceptable as long as the medication delivery is still prompt and evenly distributed.

Also presented are methods for maintaining a long operational life using a semipermeable membrane that covers a catheter lumen, where the semipermeable membrane contains a hyperosmotic or solid material. In embodiments, water from surrounding tissues may be drawn through a semipermeable membrane into a chamber fluidically connected to the catheter lumen. This fluid buildup creates a high osmotic pressure, which may be used to eject lumen deposits from the catheter lumen.

In the case that the solute of the hyperosmotic solution or the solid material is glucose (or some proxy for glucose), the saturated glucose solution, or other saturated solution, as the case may be, may be exposed to the active surfaces of the sensor and thus may also be used as a calibration fluid at appropriate intervals. In such example embodiments, the periodicity for calibration may be determined by a flexible chamber that increases in volume and pressure as water enters, and discharges through a check valve when the pressure reaches the opening pressure of the valve.

### BRIEF DESCRIPTION OF THE DRAWINGS

FIG. 1 depicts an exemplary catheter with multiple side holes to distribute a medication out of each side hole at an equal flow rate, according to one or more embodiments.

FIGS. 2A, 2B and 2C each depict an exemplary catheter that includes multiple sub-catheters to distribute a medication to independent sites and fluidics designed to provide for equal flow through each sub-catheter, according to one or more embodiments.

FIG. 3 depicts an exemplary catheter with a non-inflammatory permanent sleeve and a removeable and replaceable combination catheter/sensor, according to one or more embodiments.

FIG. 4 depicts an exemplary catheter with a paddle shaped large area diffusion component configured to be low compliance and easily implanted and removed, according to one or more embodiments.

FIG. 5 depicts an exemplary catheter with a semipermeable membrane for osmotic pressurization and/or a glucose sensor calibration solution, according to one or more embodiments.

FIG. 6 depicts an exemplary catheter with a single central lumen and distal outlet, and multiple side slits having an increasingly lower opening pressure with proximity to the distal outlet, according to one or more embodiments.

FIG. 7 depicts an exemplary catheter with a single central lumen and distal outlet, and multiple side openings that are normally closed but are each covered by only a thin membrane, according to one or more embodiments.

### DETAILED DESCRIPTION OF THE INVENTION

In embodiments, the operational life of implanted catheters and sensors may be extended so that patients will not be required to undergo excessive surgical procedures to replace catheters and sensors.

In addition to the problems described above regarding conventional catheters and medication delivery, it is also noted that the effective use of insulin delivery catheters and glucose sensors depends on the kinetics (lag) of insulin absorption from the catheter and the kinetics (lag) of glucose arrival at the sensor measurement surface. In order to automatically control glycemia, the measurement lag plus the insulin absorption lag must be minimized to fall within the glycemic excursion time due to carbohydrate consumption during a meal.

Thus, pharmacokinetic lag in the time to peak [insulin] is undesirable. The lag for insulin absorption is typically limited by the absorption from a local depot due to low surface to volume ratio of a typical depot, encapsulation of the catheter due to foreign body response, and/or slow dilution due to pooling in a depot. Insulin depots typical of subcutaneous injection sites are essentially spherical due to fact they are created from a needle point in the case of a syringe injection, or a single hole catheter tip in the case of an infusion set. This is the worst case for absorption into tissue, due to the fact that a sphere has the least surface to volume for any geometric shape. A single hole also has the potential to exhibit slow absorption if there is a foreign body reaction leading to scar tissue buildup around the catheter tip. Finally, in a spherical depot, the insulin absorption rate is limited by the process of dilution. In order to enter a capillary, insulin must break down from a hexamer to a dimer or a monomer by the process of dilution in interstitial fluid. However, in the case of a spherical depot, dilution by interstitial fluid is slow due to the limited surface area available.

Thus, exemplary embodiments of the present disclosure relate to methods for enhancing the performance and operational life of an implantable medication delivery catheter, which may also include an analyte sensor for use with a medication infusion pump. Such exemplary embodiments address various solutions to the problems of conventional delivery systems described above, due to encapsulation and lumen blockage, or the buildup of a tissue capsule around one or more sensors, and/or slow dilution due to pooling of a medication in a depot.

In embodiments, the infusion pump may be either external to the body or fully implanted. In embodiments, the catheter may be implanted in the subcutaneous tissue with the distal end of the catheter delivering into various spaces, such as, for example, blood vessels or the heart, the brain, brain ventricles and spinal spaces, the bladder, and the intraperitoneal space. In the case of insulin delivery, the preferred catheter delivery sites and sites for glucose sensing are subcutaneous tissue, blood vessels, the intraperitoneal space and the extraperitoneal space.

Medical devices of this type may also pump body fluids into a chamber for measurement of various analytes including glucose or insulin. They may also transport body fluids for other purposes such as, for example, pressure equalization for hydrocephalus using a system which pumps cerebral spinal fluid from the brain to the intraperitoneal space, or, for example, aqueous humor from the eye to treat ocular hypertension.

According to some embodiments, methods for enhancing the performance and operational life of an implantable medication catheter, which may include an analyte sensor, are presented.

The following detailed description refers to the accompanying drawings. The same reference numbers may be used in different drawings to identify the same or similar elements. In the following description, for purposes of explanation and not limitation, specific details are set forth such as particular structures, architectures, interfaces, techniques, etc., in order to provide a thorough understanding of the various aspects of various embodiments. However, it will be apparent to those skilled in the art having the benefit of the present disclosure that the various aspects of the various embodiments may be practiced in other examples that depart from these specific details. In certain instances, descriptions of well-known devices, circuits, and methods are omitted so as not to obscure the description of the various embodiments with unnecessary detail. Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed or described operations may be omitted in additional embodiments. The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used herein, including in the claims, the term “circuitry” may refer to, be part of, or include an Application Specific Integrated Circuit (ASIC), an electronic circuit, a processor, (shared, dedicated, or group), and/or memory (shared, dedicated, or group) that execute one or more software or firmware programs, a combinational logic circuit, and/or other suitable hardware components that provide the described functionality. In some embodiments, the circuitry may be implemented in, or functions associated with the circuitry may be implemented by, one or more software or firmware modules. In some embodiments, circuitry may include logic, at least partially operable in hardware.

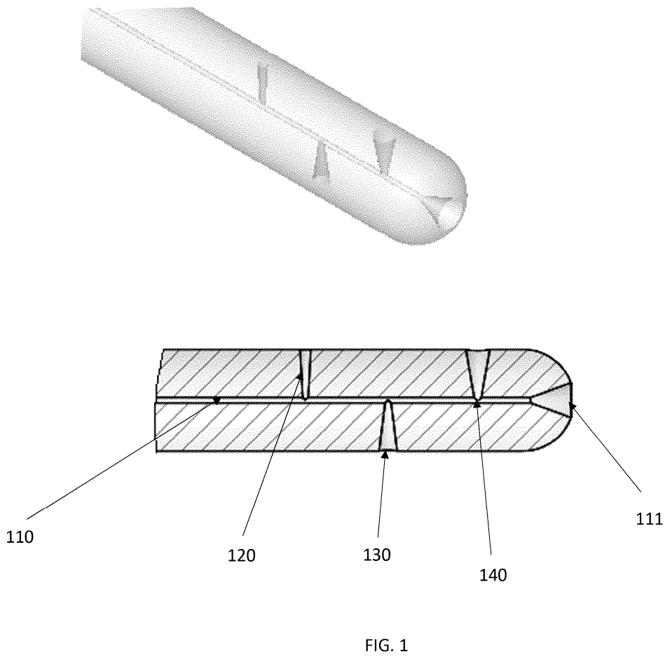

FIGS. 1-7, illustrating various embodiments, are next described.

FIG. 1 illustrates a catheter with multiple side holes designed for equal flow out of each hole. There is shown a 3D rendering of an exemplary catheter on the top of FIG. 1, and a vertical cross section of the same catheter on the bottom of FIG. 1. The example catheter has a central lumen 110, and a distal outlet 111 at a distal end of the catheter. The example catheter also has multiple side holes 120, 130 and 140, to maximize even distribution of a medication that is dispensed through the catheter and its absorption into tissue. This design takes advantage of the Poiseuille-Hagen equation for flow resistance to define catheter dimensions to achieve equal flow resistance at each opening distributed along the side wall of a catheter. As medication travels down the catheter lumen, its pressure decreases, and thus the diameter of the central lumen increases and the holes are larger. (It is noted that it may be difficult to see that the central lumen's diameter increases, as the increase it is not exaggerated in the drawing). In alternate embodiments, the central lumen diameter need not increase, and the equal flow may be achieved by only varying the size of the side holes 120, 130 and 140. Thus, side hole 130 is larger than side hole 120, and side hole 140 is the largest of all. In embodiments, the holes on the catheter 120, 130 and 140 may be distributed (at a defined pitch or inter-hole distance) so that adjacent holes will not deliver to a common depot and will not be affected by encapsulation of adjacent holes.

It is noted that a typical pump stroke may deliver on the order of 1 microliter, i.e., 1 cubic millimeter. As it emerges and forms a depot this will be expected to be no wider than a few mm. Thus, each outlet could be, for example, 10 mm isolated from its neighbor without interfering. Larger bolus deliveries would require more separation.

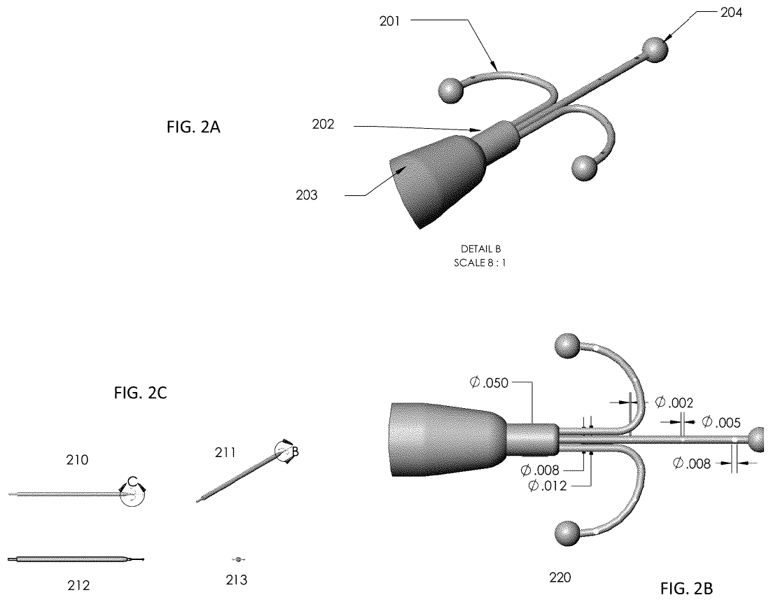

FIGS. 2A, 2B and 2C each illustrate an example catheter with multiple sub-catheters, according to some embodiments. By using sub-catheters that are widely separated in a space, such as, for example, the intraperitoneal cavity, there is a reduced likelihood that all of the sub-catheters would become encapsulated, even if one of the sub-catheters does become encapsulated. In embodiments, as shown in FIG. 2A, in order to further reduce the risk of encapsulation, the sub-catheters 201 are flexible and, for example, may each include a rounded (atraumatic) tip 204 to reduce tissue irritation. As shown in detail in FIG. 2B, the sub-catheters each include a series of side holes for large area distribution of medication to enhance the kinetics of delivery and arrival in blood. In similar fashion to the example of FIG. 1, the side holes are larger toward the distal end of the catheter to provide for equal flow rate out of each opening. In embodiments, the precise size of the openings may be calculated by the Hagen Poiseuille equation for flow in a tube and flow in an orifice.

The effect of the multiple sub-catheters and multiple side holes in each sub-catheter is to extend the operational life of the catheter by having multiple outlets. The effect of distributed delivery, especially in the subcutaneous and intraperitoneal site, is to speed up the pharmacokinetics which results in improved closed loop glycemic control.

Continuing with reference to FIG. 2A, there is shown an example catheter according to various embodiments. There is an introducer sheath 203, which may be a split peel version. As shown, the catheter system begins with a low compliance, large bore, low resistance common catheter 202, which divides into, for example, three sub-catheters 201. Each sub-catheter 201 may be a splayed preformed sub-catheter, that is straightenable within introducer sheath 203 for insertion and removal. The sub-catheters may be made of, for example, hypo tubing, Polyether ether ketone (PEEK), or polyamide. Although three sub-catheters are shown, the technique would work with just one or many sub-catheters, and, for example, seven is a good quantity for packing efficiency. In other embodiments there may be more, or less sub-catheters. In embodiments, the three sub-catheters each have a set of side holes, which, as noted, are larger toward the distal end of the catheter to provide for an equal flow rate out of each opening. The three sub-catheters 201 each end in an atraumatic tip 204, such as, for example, a rounded tip, as shown and as described above. Example, but certainly not limiting, relative dimensions are provided at the bottom right of FIG. 2, with the common catheter 202 at 0.05 units, the outer diameter of a sub-catheter 201 at 0.012 units, and the inner diameter of a sub-catheter 201 at 0.008 units. Example, but certainly not limiting, sizes for the three exemplary side holes, from proximal to distal, are 0.002, 0.005 and 0.008 units, as shown. In other embodiments there may be a greater, or lesser, number of side holes.

Continuing with the example of FIGS. 2A, 2B and 2C, in embodiments, the overall flow resistance is low due to the common catheter 202 having a large bore, and the individual sub-catheters 201 having very small bores. As noted, in embodiments, the outlet holes in each sub-catheter 201 may get progressively larger distally. In embodiments, the total flow resistance for each path is intended to be nearly equal, and each exit (outlet hole) may be a site for a microdepot, all of which may be nearly equivalent. Thus, in some embodiments, nanoliters may be output at each exit site for each sub-microliter pump pulse. Thus, the microdepots may each have a very low thickness, and a very high surface area to volume ratio.

In embodiments the example catheter may be placed in a body near vasculature—but not near fat, for optimal pharmacokinetics. Thus, in embodiments, the example catheter of FIGS. 2A, 2B and 2C may be expected to multiply the rate of absorption in the body in which it is placed, of a delivered medication.

Finally, FIG. 2C illustrates various views of the example catheter. These include, at 210 of FIG. 2C, the side view of FIG. 2B, at 211 of FIG. 2C, the perspective view shown in FIG. 2A, at 212 of FIG. 2C a side view where the sub-catheters are all in one plane that is perpendicular to the page, and at 213 of FIG. 2C a front view looking at the distal end of the example catheter where the sub-catheters are all in one plane that is perpendicular to the page.

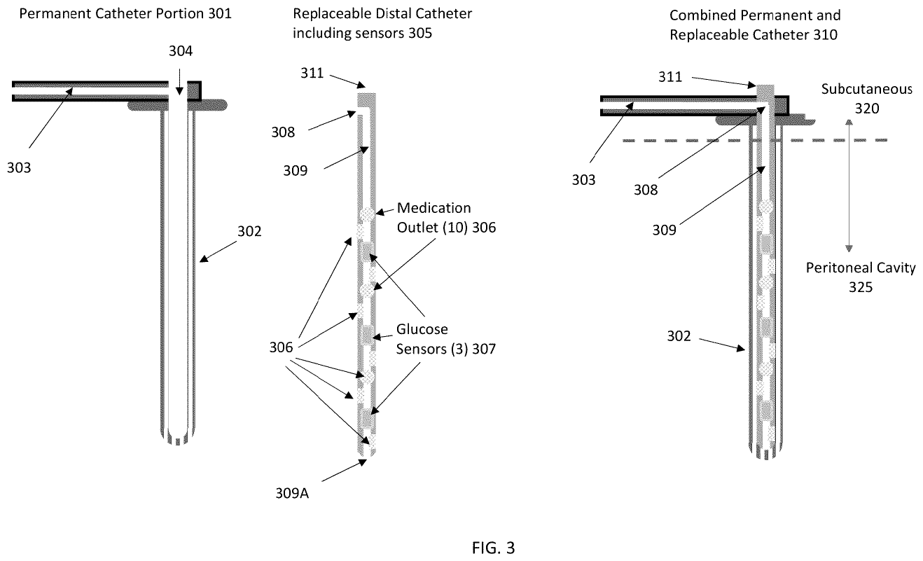

FIG. 3 illustrates a combined catheter and sensor system which is provided within a sleeve 302 that does not provoke either an inflammatory response or a foreign body response. In embodiments, the sleeve 302 may be permanently implanted in the intraperitoneal space (peritoneal cavity in FIG. 3) 325 and the catheter/sensors assembly 305 may be periodically replaced into the sleeve 302 by a surgical procedure. Because the sleeve 302 will be free floating, immersed in intraperitoneal fluid, the insulin delivered through the combined catheter system 310 disperses over a large area of tissue and is rapidly absorbed. The sensors 307 will also be able to rapidly measure for the same reason. In embodiments, sensors 307, which may be glucose sensors, are free floating, immersed in intraperitoneal fluid. In one or more embodiments, rapid pharmacokinetics and rapid sensing thus make it possible to have a robust control algorithm (which may be implemented in an ASIC within the housing containing an implantable pump) and achieve fully automatic control of glycemia.

With reference to FIG. 3, there are shown, from left to right, three images, depicting, respectively, a permanent catheter portion 301, a replaceable distal catheter including sensors 305, and a combined permanent and replaceable catheter 310. These are next described. With reference to the leftmost image, permanent catheter portion 301 has a central lumen 303 that opens, at a distal end of the permanent catheter portion, into an opening 304 in the permanent catheter portion, the opening configured to receive the replaceable distal catheter portion 305.

With reference to the central image of FIG. 3, there is shown detail of the distal catheter portion 305. Distal catheter portion 305 includes a substantially horizontal opening 308, configured to line up with the distal end of central lumen 303 of the permanent catheter portion 301, so as to create a closed fluid path through both permanent catheter portion 301, opening 308, and a central lumen 309 of replaceable distal catheter 305, as shown. Distal catheter portion 305 further includes at least two medication outlets 306, which, in the depicted example are ten, and at least one sensor, e.g., glucose sensors 307, which, in the depicted example are three. The tip or distal end of the replaceable distal catheter is open at outlet 309A to allow the medication (e.g., insulin) into, as shown in the rightmost image of FIG. 3, the peritoneal cavity 325, of a body.

Finally, with reference to the rightmost image, there is shown the combined permanent and replaceable catheter 310, where the replaceable distal catheter 305 is fully inserted through the opening 304 in the permanent catheter portion 301, and down into sleeve 302. As shown, substantially horizontal opening 308 of the replaceable distal catheter 305 is fully lined up and mated with the central lumen 303 of the permanent catheter portion 301, thus creating a closed fluid path through both permanent catheter portion 301, opening 308 and a central lumen 309 of replaceable distal catheter 305, as shown. Moreover, as shown, permanent catheter portion 301 is provided substantially horizontally (or laterally) within the subcutaneous portion 320 of a body, and the sleeve 302, and replaceable distal catheter 305 within the sleeve 302, are provided substantially vertically in the peritoneal cavity 325 of the body. At the top of the permanent catheter portion is a protruding portion 311 that sits proud above the permanent catheter portion 301, for ease of removal upon replacement.

In embodiments, the permanently implanted sleeve 302 can, for example, prevent trauma and irritation during replacement surgery to replace the replaceable distal catheter 305, thus reducing the chances for encapsulation and extending the operating life of the system. The use of multiple sensors and multiple exits provides redundancy which will extend the operational life of the catheter and in the case of the sensor, redundancy will provide reliability as well as extended life. In embodiments, the medication outlets 306 on the distal catheter 305 may be less restrictive near the tip (distal portion) of the distal catheter 305 in order to provide for equal flow rates at each exit point. Alternatively, the lumen of the distal catheter 305 may be much less restrictive than the medication outlets 306 and this will also lead to equal pressure and thus equal flow from all of the medication outlets 306.

In embodiments, the distal catheter lumen may be constructed from a coaxial composite of a non-elastomeric polymer such as polyethylene, so that there is no catheter compliance when pressure is applied and all of the fluid leaves the catheter. Likewise, the sleeve 302 is preferably close fitting so that insulin will be delivered consistently, and will not accumulate in the sleeve 302.

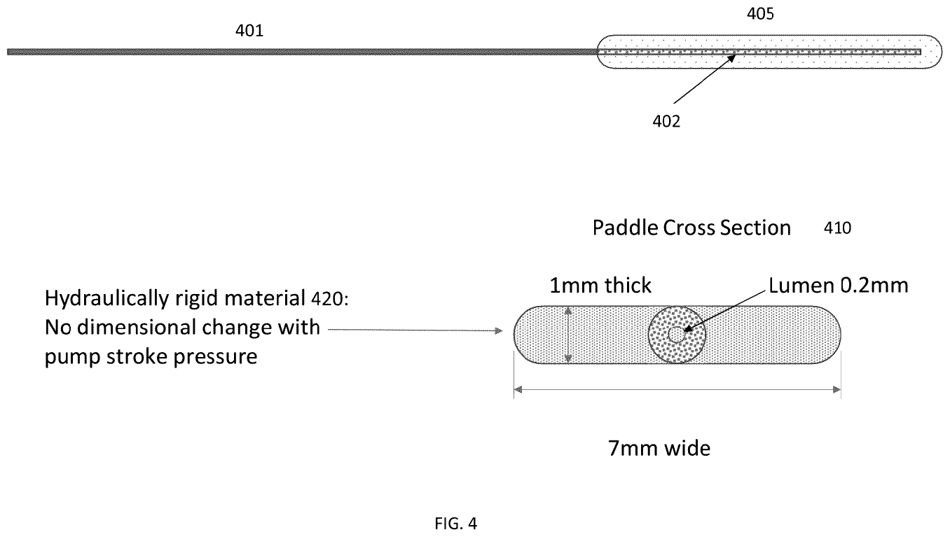

FIG. 4 illustrates a low compliance paddle shaped catheter for increased area of delivery for rapid tissue absorption in sites such as, for example, subcutaneous tissue or in the extraperitoneal site. The catheter includes a coaxial portion 401, which may be made of coaxial polyethylene and silicone, for example, and a distal paddle shaped portion 405, made of a hydraulically rigid material, which has no dimensional change with pump stroke pressure. Coaxial portion 401 extends into the paddle portion 405, as shown. The proximal composite of polyethylene and silicone of coaxial portion 401 is meant to convey the medication rapidly and completely to the distal paddle. Paddle portion 405 itself may be made of, for example, porous polyurethane, porous polyethylene, or silicone, and includes an inner lumen, surrounded by porous material. In subcutaneous tissue, insulin would be delivered from both sides of the paddle so that the maximum area of tissue is perfused. In the case of the extraperitoneal site, it is preferred to deliver from the inward facing surface to maximize intraperitoneal tissue uptake. The porous rigid paddle will distribute the insulin over a large area of tissue. The paddle is flexible, however, as noted, it does not increase in volume during delivery so that insulin will be delivered consistently and not accumulated in the paddle.

Continuing with reference to FIG. 4, there is shown a paddle cross section 410, which has exemplary, but not at all limiting, dimensional values. As shown, the paddle cross section may be 7 mm wide, 1 mm thick, and the central lumen may be surrounded by porous coaxial material, as also shown.

In embodiments, the paddle portion 405 may be coated with an anti-inflammatory material such as dexamethasone, or it be made from a material that does not provoke a foreign body response in order to extend the operational life of the catheter. In embodiments, the long, narrow paddle shape allows for convenient insertion and removal through a small diameter, mature, catheter track in tissue. The edges of the paddle may be designed to roll in during insertion and extraction.

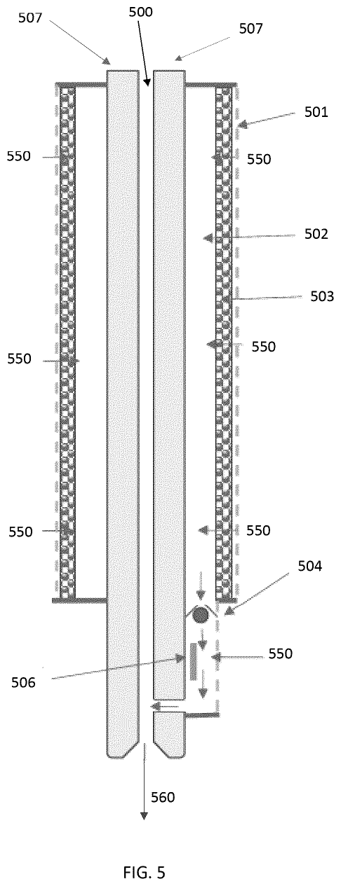

FIG. 5 illustrates an implantable medication catheter with a semipermeable membrane that acts to pressurize a saturated solution and use the pressure to dislodge a catheter obstruction, in accordance with various embodiments. Moreover, if the saturated solution is glucose, then the solution can also be used as a glucose sensor calibration solution.

In embodiments, the silicone catheter 507 has a central lumen 500 which may also be a coaxial, low compliance plastic tube made from a polyolefin or a PEEK plastic. The catheter lumen 500 is connected to a flexible chamber 502 that is filled with water. The surface of the flexible chamber 502 is a porous packet 503 of a solute, which is in contact with tissue. The outer surface of the porous packet 503 is a semipermeable membrane 501 in contact with the porous packet 503 of solute. The solute, for example could be saturated salt or sugar, or any other saccharide, in a solution where solid solute is present. The high concentration of solute inside porous packet 503 will osmotically drive water from the tissue into the solute chamber 503 and then into the flexible chamber 502, as shown by the six arrows 550 in FIG. 5, causing flexible chamber 502 to expand and develop pressure. The compliance of the flexible chamber 502 determines the volume to pressure relationship of the flexible chamber 502. The pressure that is thereby developed may be exerted directly on the fluid in the catheter tip once the pressure is significantly high thereby forcibly ejecting a lumen block such as, for example, fibrin or insulin crystals.

In embodiments, the pressure may be exerted on a check valve 504, which gatekeeps a fluid path from the flexible chamber 502 into a distal end of the silicone catheter 507, as shown. In embodiments, when the pressure reaches the opening (or cracking) pressure of the check valve 504, the valve 504 will open, and pressure will be exerted on the catheter tip volume, as shown. If there is an insulin or fibrin deposit in the tip, it will be ejected along path 560, which exits from the distal end of the silicone catheter 507, as shown in FIG. 5. Thus, when the cracking pressure of check valve 504 is exceeded, the valve opens and a bolus of fluid flows through the valve 504 and out the tip outlet. If there is an obstruction the pressurized flow can act to dislodge it. The valve then closes again, and the process repeats intermittently, repeatedly dislodging any recurring tip outlet obstruction.

Additionally, if the solute in porous packet 503 is sugar and the saturated fluid is directed over a sensor 506, then the saturated solution may be utilized as a calibration solution. Using the known concentration-output curve of the sensor, once the high value of the saturated solution is seen by the sensor, it can determine the actual concentration of any other unsaturated value. In embodiments, the periodicity of the release may be determined by four engineered parameters: (1) check valve opening pressure; (2) check valve closing pressure; (3) flexible chamber pressure vs. volume characteristic, which determines the rate of pressure build up; and (4) area of the semipermeable membrane, which determines the rate of water entry.

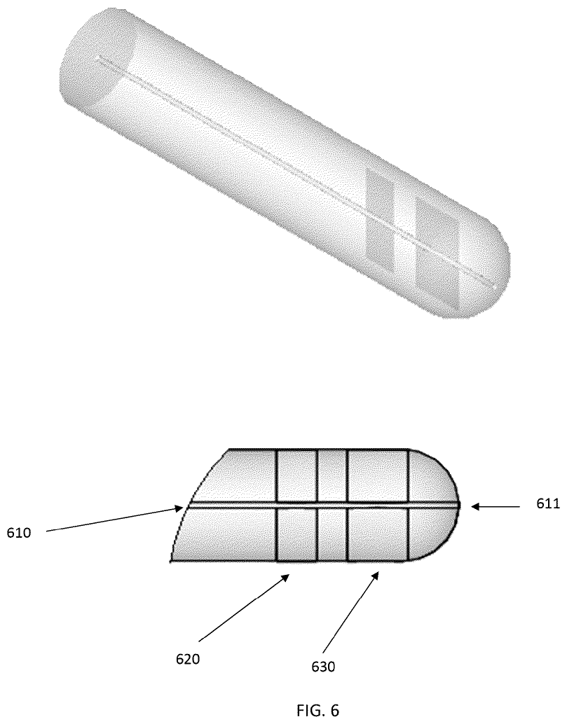

FIG. 6 depicts an exemplary catheter with a single central lumen 610 and distal outlet 611, and multiple side slits 620, 630 with increasing lower opening pressure with proximity to the distal outlet. Thus, in the event of a flow obstruction, each slit begins to open successively as pressure rises. Before then, there is virtually no flow from the slit openings and thus no biological obstruction instigated.

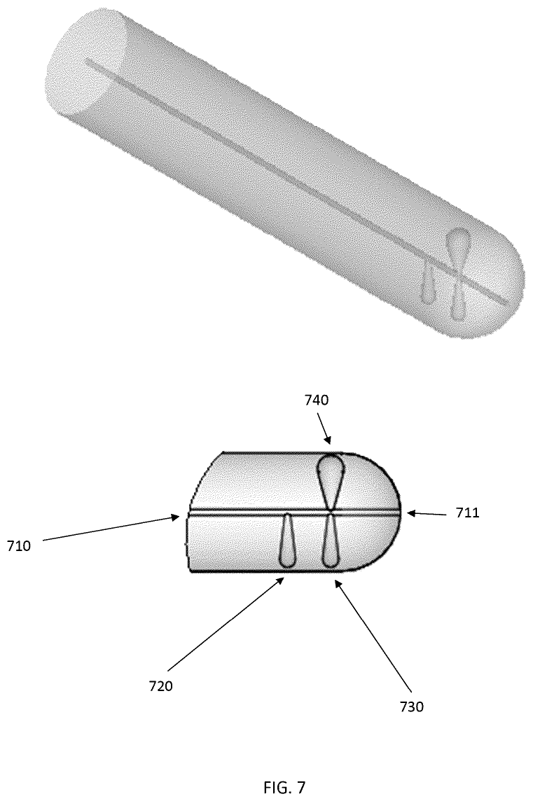

FIG. 7 depicts an exemplary catheter with a single central lumen 710 and a distal outlet, and multiple side openings 720, 730, 740 that are normally closed but that are each covered by only a thin membrane. Each membrane is designed to burst open to allow fluid delivery at a pressure that would only be achieved in the event of a distal flow obstruction. The opening burst pressure of each side opening is set by the thickness, area and material properties of the membrane.

While the foregoing is directed to embodiments of the present invention, other and further embodiments of the invention may be devised without departing from the basic scope thereof, and the scope thereof is determined by the claims that follow.

What is claimed:

1. A permanent catheter portion for use in an implanted delivery device, including:
a permanent tube with an inner lumen;
a sleeve attached to the permanent tube, the sleeve having outer coating that is at least one of: non-inflammatory or preventative of foreign body response; and
an opening in the permanent tube to receive a distal catheter portion that extends into the sleeve, such that the inner lumen of the permanent tube is fluidly connected to a distal inner lumen of the distal catheter portion.

2. The permanent catheter portion of claim 1, further comprising:
a replaceable distal catheter portion, including:
a distal inner lumen;
at least one medication outlet provided in the distal inner lumen; and
at least one chemical sensor provided in the distal inner lumen,
wherein the replaceable distal catheter portion is inserted within the opening and the sleeve of the permanent catheter portion, such as to form a closed connection between the inner lumen of the permanent tube and the distal inner lumen.

3. The permanent catheter portion of claim 1, wherein the outer coating is dexamethasone.

4. The permanent catheter portion of claim 1, wherein the outer coating is both non-inflammatory and preventative of foreign body response.

5. The permanent catheter portion of claim 1, wherein the sleeve does not allow cells to pass through it, but does allow proteins and smaller molecules to pass through it.

6. The permanent catheter portion of claim 5, wherein sleeve allows both insulin and glucose to pass through it.

7. The permanent catheter portion of claim 2, wherein the at least one chemical sensor is one of optical, electrochemical or electrochemiluminescent.

8. The permanent catheter portion of claim 2, wherein the at least one chemical sensor is a glucose sensor.

9. The permanent catheter portion of claim 2, wherein the replaceable distal catheter portion further includes a tip that protrudes above the opening in the permanent tube to sit proud above the permanent tube.

10. The permanent catheter portion of claim 2, wherein the permanent tube is provided substantially horizontally in a subcutaneous region of a body, and the replaceable distal catheter portion is provided substantially vertically in a peritoneal cavity of the body.

11. The permanent catheter portion of claim 2, wherein the at least one medication outlet is at least two medication outlets, and wherein the medication outlets are of different sizes or different restrictiveness to provide an equal flow rate from the medication outlets.

12. The permanent catheter portion of claim 2, wherein the distal inner lumen is much less restrictive than the medication outlets.

13. The permanent catheter portion of claim 2, wherein the distal inner lumen and the medication outlets are configured to have equal pressure at, and equal flow from, all of the medication outlets.

14. A method of delivering medication from an implanted catheter, comprising:
providing a permanent catheter portion in a body, the permanent catheter portion including a permanent tube with an inner lumen and a sleeve attached to the permanent tube, the sleeve having outer coating that is at least one of: non-inflammatory or preventative of foreign body response;
providing a replaceable distal catheter portion, the replaceable distal catheter portion including a distal inner lumen, at least one medication outlet provided in the distal inner lumen, and at least one chemical sensor provided in the distal inner lumen; and
inserting the replaceable distal catheter portion in the permanent catheter portion to form a combined catheter.

15. The method of claim 14, further comprising connecting the permanent catheter portion to a pump, and dispensing a medication from the pump, through the combined catheter, into the body.

16. The method of claim 14, further comprising providing the sleeve with an outer coating that is at least one of: non-inflammatory or preventative of foreign body response.

17. The method of claim 14, further comprising:
at defined time intervals, replacing the replaceable distal catheter portion with a new replaceable distal catheter portion, but leaving the permanent catheter portion unchanged.

18. The method of claim 14, wherein the medication outlets on the distal catheter portion are less restrictive near the tip of the distal catheter portion so as to provide for equal flow rates at each exit point.

19. The method of claim 14, wherein the distal inner lumen is much less restrictive than the medication outlets so as to create equal pressure, and thus equal flow, from all of the medication outlets.

20. The method of claim 14, wherein the permanent catheter portion is provided substantially horizontally in a subcutaneous region of a body, and the replaceable distal catheter portion is provided substantially vertically in a peritoneal cavity of the body.
